(19) United States
(12) Patent Application Publication
(10) Pub. No.: US 2019/0275357 A1
(43) Pub. Date: Sep. 12, 2019

(54) PORTABLE POSITIVE AIR FILTRATION DEVICE

(71) Applicants: P&B Innovations LLC, Alexander City, AL (US); AUBURN UNIVERSITY, Auburn, AL (US)

(72) Inventors: Melvin Merri PALMER, JR., Alexander City, AL (US); Jerry Dwayne BRITTON, Alexander City, AL (US); David F. DYER, Auburn, AL (US); John A. KLINNER, Auburn, AL (US); Adam L. LOGAN, Panama City, FL (US); Corey M. COX, Tupelo, MS (US); Morgan I. COOPER, Clanton, AL (US); Thomas J. BRACKIN, Opelika, AL (US); Ritu SINGH, New Delhi (IN); Christopher L. BRIGHT, Albertville, AL (US); Evans A. CARR, Birmingham, AL (US); Landon B. CARTWRIGHT, Albertville, AL (US); Sangyun GO, Auburn, AL (US); Michael T. JONES, Albertville, AL (US); Brandon M. WEAVER, Daphne, AL (US)

(73) Assignees: P&B Innovations LLC, Alexander City, GA (US); AUBURN UNIVERSITY, Auburn, AL (US)

(21) Appl. No.: 16/298,892

(22) Filed: Mar. 11, 2019

Related U.S. Application Data
(60) Provisional application No. 62/641,344, filed on Mar. 10, 2018.

(51) Int. Cl. A62B 9/00; B01D 46/42; A61B 5/00; A61B 5/1455; A61B 5/083; A61B 5/08; A61B 5/091; A62B 7/10; A62B 18/00; A62B 18/02; A62B 18/08; H04M 1/725

(52) U.S. Cl. CPC A62B 9/006; B01D 2279/40; A61B 5/7275; A61B 5/14551; A61B 5/083; A61B 5/6803; A61B 5/0015; A61B 5/0816; A61B 5/091; A61B 5/746; A62B 7/10; A62B 18/006; A62B 18/025; A62B 18/084; H04M 1/7253; B01D 46/429

(57) ABSTRACT

A portable positive air filtration device including a respiration component and a harness component. The harness component being connected with the respiration component so as to secure the respiration component against the face of a user and covering the mouth and nostril of the user. One or more fans or air-moving components, one or more filters and at least one power supply provide filtered air to the user at a positive pressure. In some examples, the device includes a mask housing, a filter, a fan and an electronics control system. One or more batteries are provided for powering the device. The electronic control system can be connected with an electronic device for real time monitoring and control.

### CROSS-REFERENCE TO RELATED APPLICATION

This application claims the benefit of U.S. Provisional Patent Application Ser. No. 62/641,344 filed Mar. 10, 2018, the entirety of which is incorporated herein by reference for all purposes.

### TECHNICAL FIELD

The present invention relates generally to the field of breathing and respiratory devices, and more particularly to portable positive air filtration devices, and to portable positive air filtration devices for connecting and communicating with one or more electronic devices.

### BACKGROUND

Positive air filtration devices are known. Typically, such devices are used in industrial applications and are very expensive and impractical for purchase by consumers. Homeowner-grade or basic face masks and respirators can be used as an alternative, however, they do not provide filtered air at a positive pressure and thus require the user's capacity for drawing air. Basic face masks are also inconvenient and irritating due to the conflicting nature of pressure change between the mask and the user. And users relying on such positive air pressure, especially during strenuous activities, would not be able to wear such masks and respirators. For example, typical portable positive air filtration devices have a separate blower and filtering system, and generally have a CPAP hose or other delivery conduit for facilitating the delivery of the filtered air to the mask. The devices are generally bulky and uncomfortable, and the mask thereof is typically full-faced wherein the entirety of the users mouth, nose and eyes are covered and protected, thereby causing the filtration device to limit a user's capabilities such as performing tasks, exercising, training, etc., practicing a sport, or other activities. It is to the provision of a portable positive air filtration device meeting these and other needs that the present invention is primarily directed.

### SUMMARY

In example embodiments, the present invention provides a portable positive air filtration device for supplying filter air to a user's mouth and nostril at a positive pressure. In example embodiments, the portable positive pressure air filtration device can be connected and communicate with at least one electronic device or smart phone.

In one aspect, the invention relates to a portable positive air filtration device including a respiration component and a harness component, the respiration component covering the mouth and nostril of a user and the harness component is provided for attachment to the respiration component so as to maintain sealed engagement of the respiration component around the mouth and nostril, the respiration component including at least one fan or air-moving component, an air filter assembly, an electronics control module electrically connected with the at least one fan, a battery supply for powering the at least one fan and electronics control module such that filtered air can be provided to the mouth and nostril of the user at a positive pressure.

In example embodiments, a seal or interface provided on at least a portion of the respiratory component for providing a seal against the user's face when the respiration component is placed thereagainst and surrounding the mouth and nostril thereof. In example embodiments, one or more one-way valves can be provided so as to permit exhausting carbon dioxide from the respiratory component. In example embodiments, an operation switch can be provided for turning on and off the electronics control module and at least one fan. In example embodiments, an electronic device can be provided for connecting and communicating with the electronics control module, wherein data of one or more components of the electronics control module or other measurements or characteristics thereof can be sent to the electronic device so as to be collected and further processed. In example embodiments, the data can be further sent to a database or server over a network. In example embodiments, the electronics control module and the electronic device communicate by Bluetooth connection. In example embodiments, one or more alerts or notifications can be displayed or emitted from the electronic device and/or the electronics control module, the alerts or notifications based on one or more measurements or data that has been determined to be outside of the normal operating conditions. In example embodiments, or more ventilation parameters can be measured and processed so as to allow for the development of a respiratory algorithm in determining the respiratory performance of a user wearing and operating the portable positive air filtration device. In example embodiments, a photoplethysmography sensor can be provided so as to utilize pulse oximetry for determining the user's present respiratory status and condition. In example embodiments, one or more components, hardware or software so as to directly measure the volume of air inhaled and exhaled from the lungs of the user. In example embodiments, the measured volume of air inhaled and exhaled from the lungs of the user can be processed so as to output the actual energy consumption by a user during a given activity.

In another aspect, the invention relates to a portable positive air filtration system including one or more portable powered respiratory devices, an electronics control module integrated in each of the one or more powered respiratory devices, and at least one electronic device for connecting and communicating with the electronics control module of the one or more powered respiratory devices. In example embodiments, each respiratory device includes a mask portion for covering the mouth and nostril of a user, a filter, a battery supply, and one or more fans for providing filtered oxygen to the mouth and nostril of the user at a positive pressure. The electronics control module includes one or more electrical components, hardware, software, sensors, a processor and/or memory.

In example embodiments, the respiratory device is portable and wearable by the user without being cumbersome and obtrusive, thereby permitting the user to wear and utilize the device in any environment and/or while performing any activity, sport or other body movements. In example embodiments, a server can be provided that is connectable with the electronic device by a network, wherein any and all data obtainable by the electronics control module can be sent to the electronic device for collection and processing, and where the data can further be sent to the server for collection and processing, wherein the server is accessible by one or more electronic devices. In example embodiments, the electronics control module and/or electronic device can output a direct ventilation measurement of respiration performance by measuring a respiration rate and a respiration volume in real time. In example embodiments, the respiration rate can be measured by use of a photoplethysmography sensor that is integrated in the respiratory device, and wherein the respiration volume can be obtained by directly measuring the volume of air that is inhaled and exhaled by the user wearing and operating the respiratory device. In example embodiments, data associated with the respiration performance including the respiration rate and respiration volume can be accessed on the electronic device, server or one or more other electronic devices. In example embodiments, one or more alerts or notifications can be emitted from the electronic device, the one or more respiratory devices, or one or more other electronic devices when a calculated value of the respiration performance falls below or exceeds values within normal operating conditions.

In another aspect, the invention relates to a positive air filtration device including a respiratory component and a harness component connected with the respiratory component, wherein at least one of the components includes one or more fans, one or more filters, one or more battery supply components, and an electronic control module including a plurality of electronic components including a processor and memory, a circuit board, and a connection component for connecting and communicating with one or more electronic devices having a processor, memory, software and an application for displaying, processing and/or calculating data that is provided from the electronic control module of the positive air filtration device, the respiratory component including an interface portion for covering the mouth and nostril of a user and wearer of the device such that filtered oxygen can be provided to the mount and nostril of a user at a positive pressure.

In example embodiments, the respiratory component and the harness component are integrally formed together. In example embodiments, the respiratory component and the harness component are separate pieces and removably or permanently attached to the respiratory component. In example embodiments, the harness component is adjustable. In example embodiments, the connection component is a Bluetooth component.

### BRIEF DESCRIPTION OF THE DRAWINGS

FIG. 1 shows a portable positive air filtration device being worn by a user according to an example embodiment of the present invention, the user holding an electronic device that is connectable with the portable positive air filtration device.

FIG. 2 shows a front perspective view of the portable positive air filtration device of FIG. 1.

FIG. 3 shows a rear view of the portable positive air filtration device of FIG. 2.

FIG. 4 shows the portable positive air filtration device of FIG. 2, wherein the filter assembly is removed therefrom to show hidden components of the portable positive air filtration device.

FIG. 5 shows a rear perspective view of a housing component of the portable positive air filtration device of FIG. 3.

FIG. 6 shows a side view of the housing component of the portable positive air filtration device of FIG. 5, further showing various filter assemblies that can be used therewith according to example embodiments of the present invention.

FIG. 7 shows a schematic of the portable positive air filtration device of FIG. 2 connected with an electronic device, a network, server and other electronic device according to example embodiments of the present invention.

FIG. 8 shows a top plan view of a portable positive air filtration device according to another example embodiment of the present invention.

FIG. 9 shows a perspective view of a portable positive air filtration device according to another example embodiment of the present invention.

### DETAILED DESCRIPTION OF EXAMPLE EMBODIMENTS

The present invention may be understood more readily by reference to the following detailed description of example embodiments taken in connection with the accompanying drawing figures, which form a part of this disclosure. It is to be understood that this invention is not limited to the specific devices, methods, conditions or parameters described and/or shown herein, and that the terminology used herein is for the purpose of describing particular embodiments by way of example only and is not intended to be limiting of the claimed invention. Any and all patents and other publications identified in this specification are incorporated by reference as though fully set forth herein.

Also, as used in the specification including the appended claims, the singular forms “a,” “an,” and “the” include the plural, and reference to a particular numerical value includes at least that particular value, unless the context clearly dictates otherwise. Ranges may be expressed herein as from “about” or “approximately” one particular value and/or to “about” or “approximately” another particular value. When such a range is expressed, another embodiment includes from the one particular value and/or to the other particular value. Similarly, when values are expressed as approximations, by use of the antecedent “about,” it will be understood that the particular value forms another embodiment.

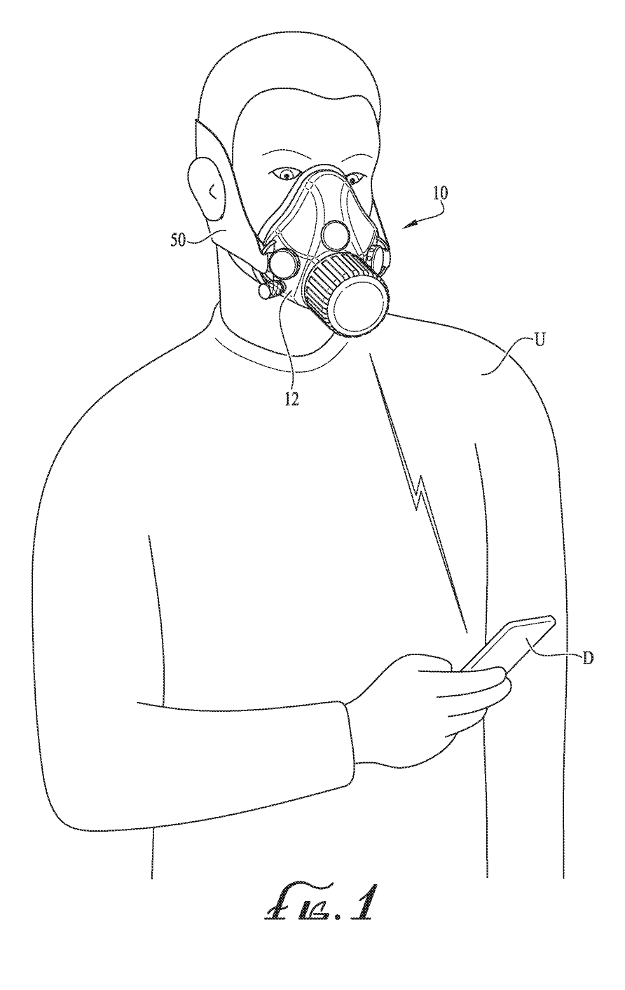

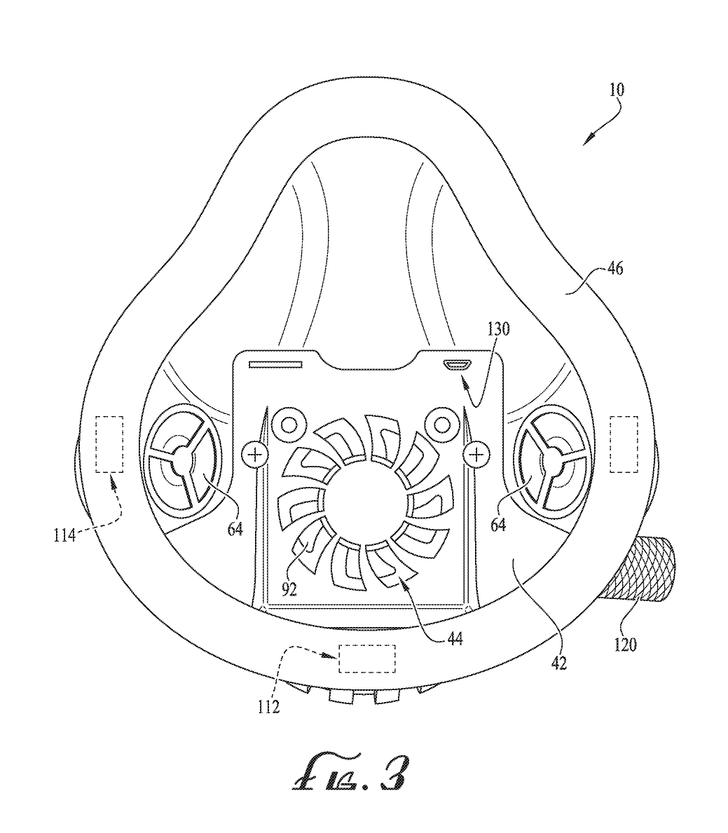

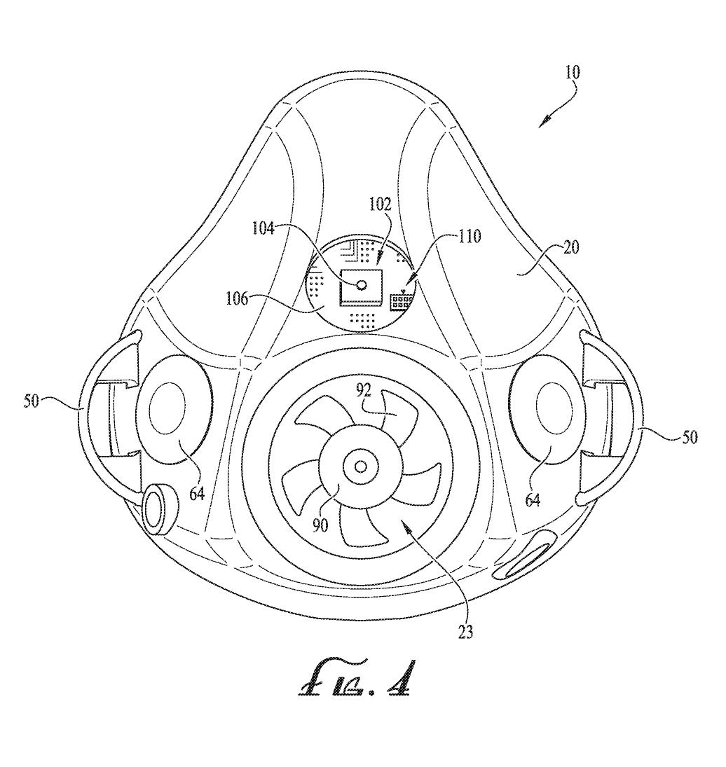

With reference now to the drawing figures, wherein like reference numbers represent corresponding parts throughout the several views, FIGS. 1-7 show a portable positive air filtration device 10 according to one example embodiment of the present invention. As depicted in FIG. 1, the device 10 is configured for attachment to a face of a user U, for example, to be worn by the user U as desired and filter the air or oxygen inhaled (e.g., breathed) by the user U. In example embodiments, the device 10 is powered such that a fan or blower 90 (see FIGS. 3-4) outputs filtered air within the device 10 to provide a continuous positive flow of air therein, thereby assisting the user U in the inhalation of the filtered air. In example embodiments, the device 10 is preferably configured for only covering the mouth and nose of the user's U face, for example, wherein the eyes of the user remain uncovered and exposed to the elements. In example embodiments, the device 10 is preferably sized and shaped so as to allow for the use of goggles, protection glasses or other shields or protectors while wearing the mask. In example embodiments, the device 10 is preferably comprised of one or more assembled components, which when connected together form a single and unitary device, for example, a mask or respirator component 12 and a strap or harness component 50 connected with the respirator component 12 for maintaining attachment of the respirator component 12 to the face of the user U. Thus, the device 10 of the present invention is preferably portable and wearable by the user U without being cumbersome and obtrusive, thereby permitting the user U to wear and utilize the device 10 in any environment and/or while performing any activity. For example, the user U may wear the device 10 during one or more strenuous or laborious activities, while performing manual labor, or for example, while exercising or partaking in various forms of athletic training.

According to example embodiments of the present invention, the device 10 can communicate with an electronic device D. For example, according to one example embodiment, any and all data that is obtainable or recorded by the device 10 can be sent to the electronic device D. In example embodiments, the electronic device D is a smart phone or device, or for example, a tablet or other smart device capable of communicating with other devices over one or more signals and/or networks and/or hosting/receiving stations or radios, or other wireless communication systems or methods. For example, according to one example embodiment, the device 10 is wirelessly connected to the electronic device D via a Bluetooth signal. Accordingly, by connection of the device 10 and the electronic device D via a Bluetooth connection, data captured by the device 10 can be sent therefrom and accepted or received by the electronic device D. In another way, the electronic device D can send data to the device 10, for example, whether it be a software update for one or more components of the device 10, or for example, to provide remote operation of the electronic device 10. Optionally, other means of connection such as WiFi, infrared (IR), radio (RF) or other means for wirelessly connecting two electronic components and/or devices can be provided as desired, for example, such that data being collected by the device 10 can be sent directly to the electronic device D in real time (and data can also be sent from the electronic device D to the device 10 in real time).

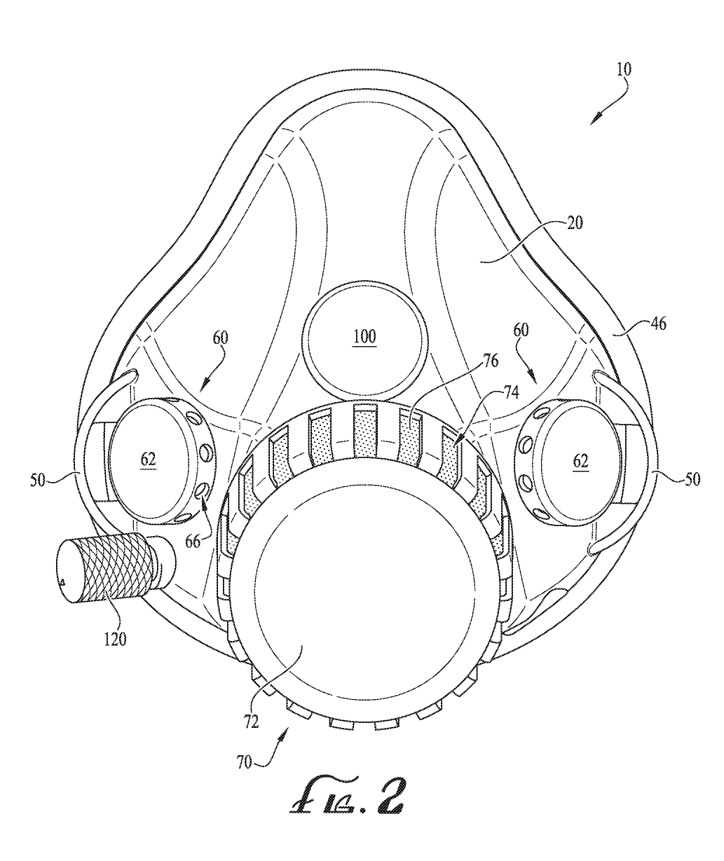

As depicted in FIG. 2, the device 10 generally comprises a mask interface or housing component 20, one or more exhalation gate or one-way air flow valve assemblies 60, a filter assembly 70, a blower or fan 90 and an operation switch 100. In example embodiments, the housing component 20 is generally shaped to conform to the face of a person, for example, comprising a triangular-like shaped shell defining a recessed or concaved interior portion 21 and having a generally central upper portion for fitting around the nose of the user U and a wider lower base portion for fitting around the mouth of the user U. A rear edge 22 of the housing component 20 provides for attachment of a seal or face interface 46, for example, which is generally flexible and resilient to seal and conform to various face profiles. In example embodiments, the seal member 46 can be comprised of one or more synthetic and/or natural materials, for example, one or more generally flexible and resilient materials, for example, such as a silicone, gel, foam, plastic, composite, or other available materials as desired. According to example embodiments, the seal member 46 can be generally mechanically clipped or attached to the housing 20, or for example, one or more adhesives or other bonding methods can be provided. According to another example embodiment, the seal member 46 is integral with the housing 20 for example rather than being a separate piece that is connected with the housing 20. According to example embodiments, the seal member 46 can comprise a surface treatment to provide for a sufficient seal against the user's U face. Furthermore, the harness component 50 can preferably provide adjustability so as to tighten the respiratory component 12 against the face of the user U. According to one example embodiment, a pair of attachment rings 50 are provided with the housing 20 for facilitating attachment of the harness component 50 thereto.

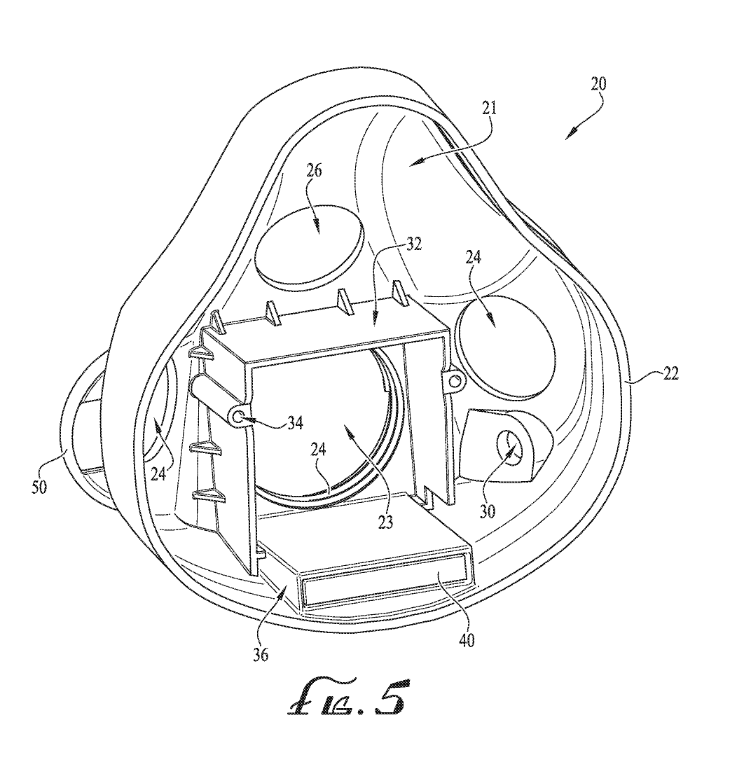

In example embodiments, the valve assemblies 60 are provided to permit the user to expel carbon dioxide (CO₂) out of the respiratory component 12, for example, such that the CO₂ exhaled from the user is permitted to pass through the valve assemblies 60 (e.g., exiting therefrom) while not permitting any unfiltered oxygen from entering therethrough in a generally opposite direction. In example embodiments, the housing 20 comprises two spaced-apart openings 24 that are configured for securely receiving the valve assemblies 60 (see FIG. 5). According to example embodiments, each of the valve assemblies 60 comprises an outer housing 62, an umbrella valve component 64 (see FIGS. 3-4), and one or more openings 66 formed through the housing 62 for permitting the CO₂ that passes by the umbrella valve to exit from the housing 62. According to other example embodiments, other valve assemblies such as Belleville, X-fragm, dome with backup seal, or other valves, one-way flow valves, components or assemblies can be provided as desired. In example embodiments, the valve assemblies can be permanently or removably mounted with the openings 24 of the housing 20. In some example embodiments, one or more mechanical snaps or couplings can be provided for attachment to the housing 20. In other example embodiments, one or more adhesives or glue can be provided for attachment to the housing 20. Preferably, the umbrella valve component 64 is substantially flexible and resilient, for example, to maintain a seal and prevent oxygen from entering therethrough but permit CO₂ expelled from the user U to pass therethrough and exit to the environment.

In example embodiments, the filter assembly 70 is preferably provided for attachment to at least a portion of the housing 20 and filter the oxygen that is drawn into the respiratory component 12 and inhaled by the user U. In example embodiments, a central opening 23 of the housing 20 (see FIG. 5) comprises one or more threads 23a for attachment of the filter assembly 70. According to one example embodiment, a threaded connection is provided for securely connecting the filter assembly 70 with the opening 23. In other example embodiments, one or more interengagement members, couplings, connectors, clips, snaps or other attaching-components are provided so as to secure the filter assembly 70 with the housing 20. According to some example embodiments, one or more o-rings, washers or other sealing components can be provided such that the filter assembly 70 fully seats and seals with the housing 20. In example embodiments, the filter assembly 70 comprises a housing 72, one or more openings or pass-thru channels 74 formed in the housing 72, and a filter component 76 positioned within the housing 72. According to some example embodiments, the housing 72 comprises a reusable cartridge and the filter component 76 can be replaced as desired. According to example embodiments (as will be described below), the filter assembly can preferably be sized and shaped as desired, for example, comprising a desired length, width and filter component 76.

In example embodiments, the fan 90 is preferably provided for attachment to the housing 20 so as to draw oxygen from the external environment and through the filter assembly 70 for providing a positive flow of filtered air for inhalation by the user U. In example embodiments, the housing 20 comprises a fan mount housing 32 (see FIG. 5), for example, which is generally aligned with the opening 23 so as to allow for connection of the fan 90 thereto (see also FIG. 4). In example embodiments, the fan 90 comprises an impeller having one or more blades 92 that are generally positioned about a circular array such that rotation of the impeller causes air to be drawn into the respiratory component 12. In example embodiments, the fan 90 is preferably of the type that is capable of high RPM and low power draw, for example, such that a positive pressure of air flow is provided to the user U while requiring minimal power (e.g., so as to conserve the life of the power supply—battery 40). According to one example embodiment, a 12 volt Mabuchi fan is provided for use with the device 10. In example embodiments, the fan is rated for up to 16 volts and a max RPM of 12,500. In typical example embodiments, the fan 90 is generally rated to comprise a max RPM ranging from between about 9,000 RPM to about 35,000 RPM, for example, between about 12,000 RPM to about 25,000 RPM according to one example embodiment. According to alternate example embodiments, the fan 90 can preferably be rated to comprise a max RPM of 22,000 or more.

According to example embodiments, the fan 90 of the present invention delivers a desired volumetric flow rate and positive pressure. In example embodiments, the volumetric flow rate of the fan 90 is generally between about 1.5-6.5 cubic feet per minute (CFM), for example, between about 2.5-4.5 CFM according to one example embodiment. In example embodiments, the positive pressure is generally between about 1.5-6 in H₂O, or for example about 2.36 in H₂O according to one example embodiment. According to another example embodiment, the device delivers a positive pressure of between about 2-8 in H₂O, for example about 4 in H₂O according to one example embodiment.

In example embodiments, the fan 90 is of the type comprising a variable control feature, for example, such that the amount of power that is supplied to the fan can be adjusted, and thus, thereby control the speed of the fan. Thus, according to example embodiments, the fan speed can preferably be adjusted as desired by the user, for example, so as to provide a desired positive pressure air flow within the respiratory component 12. In example embodiments, a pulse-width modulation (PWM) controller can be provided so as to adjust the amount of power provided to the fan 90, and thus, allow for variable speeds. In example embodiments, a control knob 120 (see FIGS. 2-3) can be provided for manual adjustment of the fan speed. As depicted in FIG. 5, a secondary control opening 30 is provided through a portion of the housing 20 for accommodating mounting of the knob 120 thereto. Preferably, manual rotation of the knob 120 provide for adjustment to the speed of the fan 90. According to another example embodiment, a digital fan component, or for example, a counter-rotating fan can be provided for use with the device 10. According to other example embodiments, other available fans or air moving devices can be provided as desired. According to one example embodiment, two or more fans 90 can be provided, for example, rather than a single fan.

In example embodiments, the operation switch 100 preferably provides for operating the device 10, for example, so as to permit the device 10 to be turned on so as to rotate the fan 90 and draw oxygen through the filter assembly 70 and further through one or more openings 44 of an inner liner 42 (see FIG. 3) to provide a desired positive pressure air flow within the respiratory component 12. According to example embodiments, the inner liner 42 is preferably sized and shaped to be fitted within the concave portion 21 of the housing 20, for example, to substantially cover and conceal one or more of the components mounted thereto (as will be described below). According to example embodiments, one or more fasteners can be configured for engaging receivers 34 within the concave portion 21, for example, so as to secure the inner liner 42 to the concave portion 21 of the housing 20. In other example embodiment, the liner 42 and the housing 20 can preferably be connected by one or more clips, couplings, connectors, clasps, fasteners, adhesives, welds, and/or other available connecting components. As described above, the liner 42 comprises the one or more openings 44 for permitting filtered air to pass therethrough for inhalation by the user U.

According to one example embodiment, the operation switch 100 is generally positioned near an opening 26 of the housing 20 (see FIG. 5). In example embodiments, the operation switch 100 is in the form of a push button, for example, comprising an actuatable portion that is generally pressed or actuated so as to enable or disable power to the device 10. In example embodiments, the operation switch 100 comprises a depressible membrane or member covering the opening 26, and which is exposed and positioned on the housing 20 such that a user U can easily access during use. As shown in FIG. 4, a button or switch component 102 comprising a module 104 is mounted to a circuit board 106 that is positioned generally near the opening 26 such that pressing on the depressible membrane contacts at least the module 104, and thus, actuating the module 104 causes either turning on or turning off the device 10. According to example embodiments, the circuit board 100 is positioned and connected within the concave portion 21 of the housing 20 and the switch component 102 is mounted to the circuit board 106 so as to be generally centrally positioned within the opening 26 such that pressing against the membrane further causes contact with the switch component 102.

In example embodiments, the circuit board 106 is preferably a component of the device's 10 electronic control system, for example, such that the electronic components of the device 10 are preferably properly eclectically connected together to function as desired. In example embodiments, the electronic control system can comprise the circuit board 106, a processor and memory, a Bluetooth component 110, a battery supply 40, one or more sensors 112, 114, the fan 90, the operation switch 100 (and switch component 102), a control knob 120, a charging outlet 130. According to additional example embodiments, one or more additional components such as a GPS component, an accelerometer, gyro, etc. can be provided with the electronic control system as desired.

As described above, the device 10 is preferably configured for communicating with the electronic device D, and vice-versa, for example, whereby the Bluetooth component 110 is connectable with a Bluetooth component of the electronic device D, or for example, the Bluetooth component 110 is connectable with a Bluetooth component 110 of another device 10. According to another example embodiment, the device 10 is capable of communicating with devices 10 or electronic device D over one or more signals and/or networks and/or hosting/receiving stations or radios, or other wireless communication systems or methods. For example, according to one example embodiment, the device 10 is wirelessly connected to the electronic device D via a Bluetooth signal (as described above). Accordingly, by connection of the device 10 and the electronic device D via a Bluetooth connection, data captured and/or collected by the device 10 can be sent therefrom and accepted or received by the electronic device D. In another way, the electronic device D can send data to the device 10, for example, whether it be a software update for one or more components of the device 10, or for example, to allow for remote operation of the device 10 by accessing controls on the electronic device D. Optionally, other means of connection such as WiFi, infrared (IR), radio (RF) or other means for wirelessly connecting two electronic components and/or devices can be provided as desired, for example, such that data of the device 10 can be sent directly to the electronic device D in real time (and data can also be sent from the electronic device D to the device 10 in real time).

In example embodiments, the battery 40 is housed within a battery housing 36 that is formed within a portion of the housing 20 (see FIG. 5). According to one example embodiment, the battery comprises a lithium ion type battery comprising a capacity of 3400 mAh and a voltage of about 3.6. In example embodiments, the battery 40 is preferably sufficient so as to power the device 10 for between about 2 hours to about 8 hours of run time. According to one preferred embodiment, the battery 40 is preferably sufficient to power the device 10 for between about 3 hours to about 7 hours, for example, between about 5-6 hours according to one preferred embodiment. In other example embodiments, the battery 40 can preferably be configured so as to provide a desired run time. According to example embodiments and as described above, the description of powering the device 10 or run time considers the fan running at max RPM. Accordingly, powering the device 10 where the fan speed is less than the max RPM can allow for extended battery life beyond the ranges as described herein.

According to one example embodiment, one or more external power supply components can be provided so as to improve the battery life and permit the device 10 to be powered (e.g., and thus operate) for a longer period of time beyond the above-described run time estimations. For example, in some example embodiments, a separate power supply or battery can be electrically connected with the device 10, for example, such that even when the life of the battery 40 is substantially or entirely reduced (e.g., the battery is dead and needs recharging), the external power supply can be connected with the device 10 such that the device 10 can still properly function as intended. According to one example embodiment, one or more connectors, straps, clips, couplings, mounts, or other holding or containment components can be provided for removable attachment of the power supply to one or more portions of the device 10. In one example embodiment, a sleeve is formed within a portion of the harness component 50 so as to receive a separate power supply. Thus, even with the separate power supply electrically connected with the device 10, the device 10 remains a single unitary device. In alternate example embodiments, the separate battery supply can be configured so as to mount to other portions of the device 10 or can be configured for removable attachment to the user U.

In example embodiments, the separate battery supply can be configured for electrically connecting with the device by a wired connector or electrical cable. In example embodiments, one or more power supply ports can be provided with the device 10 so as to easily allow for electrically connecting the separate power supply with the device 10. In other example embodiments, one or more electrical terminals can be provided with the device 10 such that the separate battery supply becomes electrically connected with the device 10 when it is removably mounted thereto. In other example embodiments, one or more housings, cartridges, compartments, pockets, sleeves, or other container-like components can be provided with the device 10 so as to facilitate easily mounting, connecting, removing, etc. of the separate power supply with the device 10.

In example embodiments, the charging outlet 130 is provided in an interior portion 21 of the housing 20 (see FIG. 3). In example embodiments, the outlet 130 preferably provides for connecting an electrical cable or wire, for example, such that the battery supply 40 can be recharged by a standard power outlet. According to example embodiments, a first end of the electrical cable is connected to the outlet 130 and a second end of the electrical cable is connected to a standard power outlet, for example, a traditional 120V power outlet. In some example embodiments, an AC/DC converter can be provided. According to some example embodiments, the second end of the electrical cable comprises a USB connector, for example, such that the USB connector can be electrically connected with a USB port so as to allow for recharging of the battery supply 40. According to other example embodiments, one or more ends of the cable can comprise a micro-USB cable, or for example, can comprise other available electrical connectors as desired. According to another example embodiment, the outlet 130 can be located on any other portions of the device. For example, according to one embodiment the outlet 130 is provided on an external portion of the device 10 and a charging dock (e.g., that is electrically connected to the standard power outlet) is connectable with the outlet 130. Thus, according to some examples, the device 10 can be docked or generally placed on the charging dock to complete the electrical connection therebetween and permit charging of the battery supply 40. According to another example embodiment, a charging component utilizing induction charging capabilities can be provided, for example, such that merely placing the device 10 atop the charging component causes charging of the battery supply 40.

As depicted in FIG. 3, the seal member 46 can comprise one or more sensors embedded therein, for example, so as to allow for obtaining real-time data associated with the user U. In example embodiments, one or more sensors 112, 114 can be positioned on at least a portion of the seal member 46 so as to provide contact or engagement with the face or skin of the user U when the device 10 is worn. According to one example embodiment, the one or more sensors 112, 114 collect data so as to obtain information regarding the user's U heart rate. In other embodiments, the one or more sensors 112, 114 can be configured for collecting data pertaining to various other characteristics of the user U wearing the device 10. In example embodiments, the one or more sensors 112, 114 can be a photoplethysmography (PPG) sensor. In another example embodiment, the one or more sensors 112, 114 can be an electrocardiography (ECG) sensor. In alternate embodiments, the one or more sensors 112, 114 can be chosen as desired. In some example embodiments, the one or more sensors 112, 114 are embedded within at least a portion of the seal member 46. In other example embodiments, the one or more sensors 112, 114 can be mounted and positioned as desired, for example, in one or more locations on the respiratory component 12 or harness component 50 as desired.

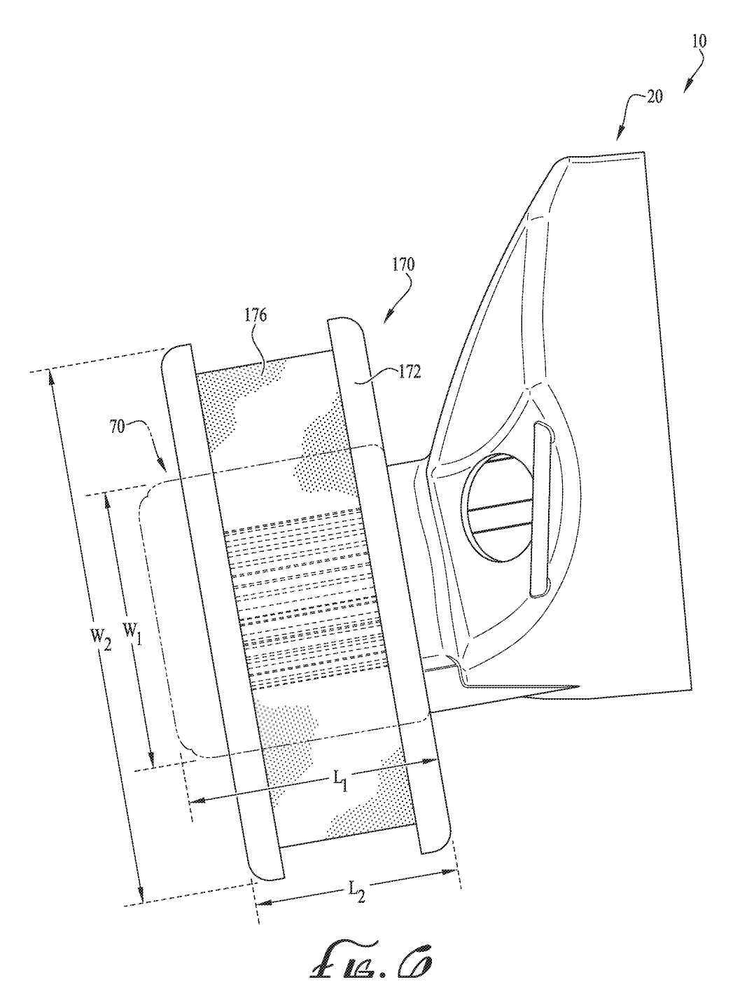

As depicted in FIG. 6, the device 10 can preferably comprise a desired filter assembly 70, for example, comprising a desired size (e.g., length and width), shape and filter component 76. In example embodiments, the filter component 76 of the present invention preferably provides for airborne particulate filtration of a minimum of 95%. According to example embodiments of the present invention, the filter component 76 can be of various ratings, for example, a rating such as N95, N99, N100, R95, R99, R100, P95, P99 or P100. According to example embodiments, the letter “N” is classified as not oil resistant, the letter “R” is classified as resistant to oil, and the letter “P” is classified as oil proof. Preferably, a filter having one of the ratings of the classification letters and numbers listed above can be provided for use with the device 10. According to one example embodiment, the filter component comprises a foam-like open cell filter material, for example, which generally comes in the form of a cylindrical filter member for assembly with the housing 72 (or other reusable cartridge that is connectable to the housing 20). According to another example embodiment, the filter component 76 comprises a paper material or other paper-like filter material. According to some example embodiments, the filter component 76 comprises an assembly of filter materials, for example, which can be housed together in an assembled filter component 76 for insertion and removal from the filter cartridge, filter housing or for direct attachment to the respiratory component 12 or housing 20 thereof. According to another example embodiment, the filter assembly 70 comprises an assembled filter cartridge, for example, so as to make the replacement process easy and user-friendly. According to some example embodiments, the filter cartridge is generally in the form of a canister for attachment to at least a portion of the device 10. According to some example embodiments, other filter types, for example, a pancake-type filter, filter discs, prefilters, and/or one or more other filter components and types can be provided for use with the device 10.

According to additional example embodiments of the present invention, the filter component 76 can be configured for protection against organic vapors such as solvents, or for example, for protection against toxic dusts such as lead or asbestos. According to some example embodiments, filter cartridges 76 can provide for protection against organic vapors, chlorine, hydrogen chloride, sulfur dioxide, hydrogen sulfide, hydrogen fluoride, chlorine dioxide, ammonia, methylamine and/or formaldehyde. Optionally, filter cartridges capable of filtering other harmful gasses, vapors, chemicals, etc. can be provided with the device 10. According to some example embodiments, as will be described below, information pertaining to the filter's pore size can be provided to the user on an electronic device D, and for example, obtain and analyze the present atmospheric conditions at the device's D location to recommend filter pore size or type.

For example, according to one example embodiment the housing 20 can comprise the filter assembly 70 (comprising the filter housing 72, openings/passageways 74, and filter component 76) and define a length L1 of between about 1.5-3 inches, for example, about 2 inches according to one example embodiment and a width W1 of between about 1-3 inches, for example, about 1.7 inches according to one example embodiment (see FIG. 6). According to another example embodiment, a filter assembly 170 (comprising filter housing 172 and filter component 176) can optionally be provided for attachment and use with the device 10 (and housing 20 thereof), for example, and define a length L2 of between about 1-4 inches, for example, about 1.75 inches according to one example embodiment and a width W2 of between about 1-6 inches, for example, about 4.5 inches according to one example embodiment (see FIG. 6).

As described above, the device 10 of the present invention can preferably comprise an electronic control system so as to provide for connecting (and communicating) with at least one electronic device D. For example, according to example embodiment of the present invention, the at least one electronic device D comprises hardware and software that can connect and communicate with the device 10. According to example embodiments, the electronic control system (and components thereof) generate data pertaining to one or more measurements, temperatures, humidity, pressures, air flow, air characteristics, ventilation parameters, or other characteristics of one or more of the components.

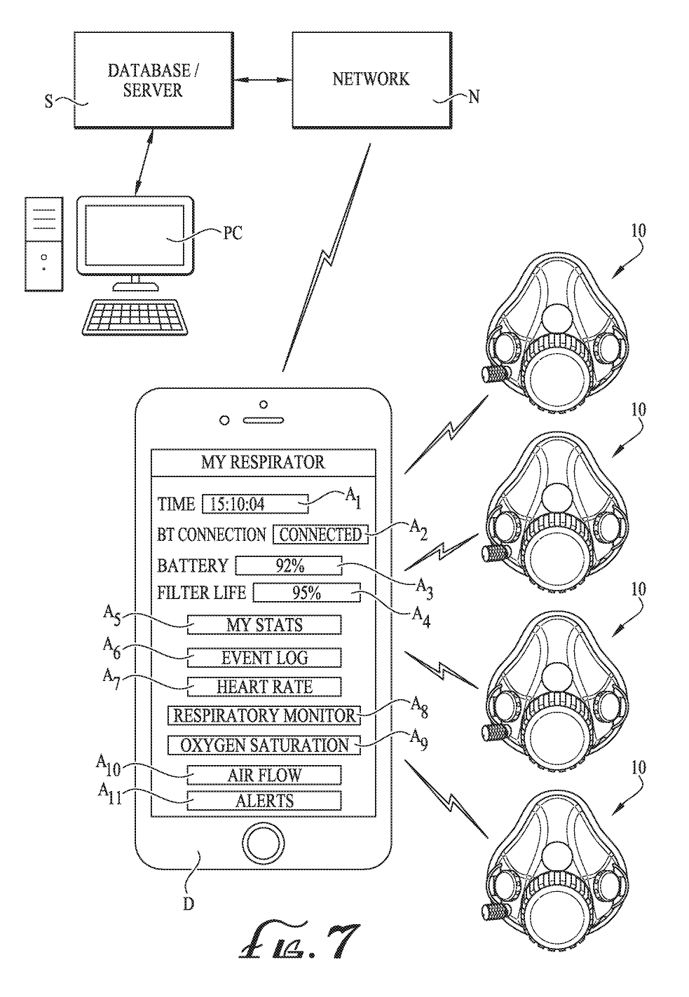

As depicted in FIG. 7, the at least one electronic device D is connectable (and communicates) with at least one device 10, for example, about four devices 10 according to one example embodiment. According to one example embodiment, the electronic device D comprises software or a program or application for interfacing with the at least one device 10. According to example embodiments, a display screen of the electronic device D displays a plurality of characteristics and data pertaining to the at least one device 10. In example embodiments, the software displays the present time A1, the status of the Bluetooth connection A2, the battery status A3 and the air filter life remaining A4.

Further, one or more additional characteristics/measurements/data sets can be sent to the electronic device D in real time, for example, to be collected and processed to provide additional information such as a “My Stats” page A5, an “Event Log” page A6, a “Heart Rate” page A7, a “Respiratory Monitor” page A8, an “Oxygen Saturation” page A9, an “Air Flow” page A10, and for example a “Alerts” page A11. In example embodiments, the pages A5-A11 can be individually entered (or accessed) for viewing the corresponding data relative to the particular page.

According to one example embodiment, the software of the electronic device D monitors the user's CO₂ level and acts as a failsafe to alert the user if it ever reaches an unsafe level. For example, by obtaining data from the device 10 pertaining to the filter blockage, respiration rate, respiratory volume, temperature and humidity levels, the user's U CO₂ level can be obtained and accessible in real time on the electronic device D (or server S). According to example embodiments, collecting and processing the data obtained from the device 10 to calculate the user's U CO₂ level can provide the user U with assurance that they are working/using the device 10 in a safe environment and that the mask is working properly and keeping them safe.

According to example embodiments, when the collected data is processed and there is an indication of one or more values or calculations outside of the normal operating range, an alert can be provided so as to inform the user of the out-of-normal-operating-range values. In some example embodiments, the alert is output from the electronic device D, or for example, the alert can be output from the device 10 and/or the electronic device D. In some example embodiments, one or more alerting indicators or components such as audio, vibration and/or other feedback can be integral with the device 10, for example, such that the user U wearing and operating the device 10 is immediately informed. In other example embodiments, one or more alerts can also be provided or output from one or more personal computers PC or other electronic devices that are accessing the data from a server S (as will be described below).

According to example embodiments, the data received by the electronic device D is further sent or uploaded over network N to a database or server S where it is securely stored. According to example embodiments, the data saved on the server S can be accessed from an electronic device or other personal computing device PC, for example, which may be in same or different location relative to the electronic device D (and at least one device 10 connected therewith). For example, according to one example embodiment, an administrator or other user can access the data from the at least one device 10 from the personal computer PC while a user U is wearing and operating the device 10, for example, such that the data is generally uploaded to the server S and accessible by the personal computer PC in real time. In other example embodiments, at least some delay may be present from the time the data is collected on the electronic device D to when the same data is uploaded and accessible on the server S.

In example embodiments, the software of the electronic device D preferably comprises one or more features thereon for providing remote control operation of the device D. According to one example embodiment, the fan 90 can be controlled or the speed thereof can be infinitely adjusted (e.g., rather than manual adjustment of the knob 120), the device 10 can be powered on or off (e.g., rather than requiring manual manipulation of the operation switch 100), and/or other components/features/etc. of the device 10 can be controlled via the electronic device D. According to one example embodiment, each device 10 is connected and communicates with a separate electronic device D, for example, which can be carried and accessed by the user U during the use and operation of the device 10. According to another example embodiment and as depicted in FIG. 7, a plurality of devices 10 can be connected and communicate with the same electronic device D. In such embodiments, the software is preferably configured so as to provide a plurality of individual pages associated with each of the connected devices 10. Preferably, regardless of whether one or more devices 10 are connected and communicating with the electronic device D, the data sent to the electronic device D can be sent in its entirety to the server S to be saved.

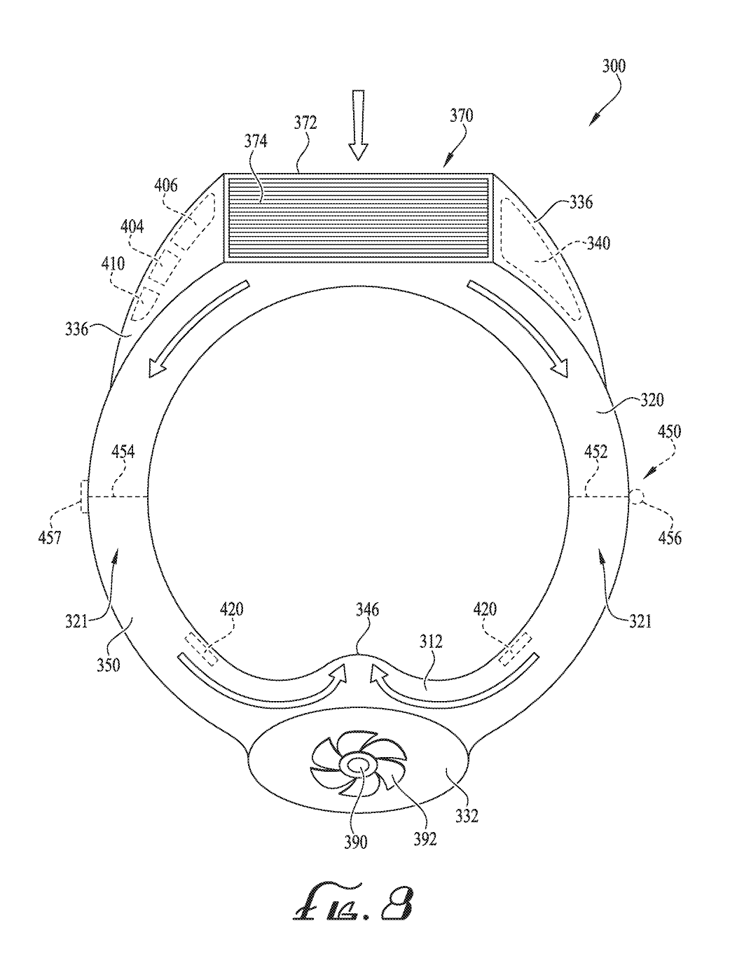

According to additional example embodiments, the portable positive air filtration device can be preferably configured as desired. For example, FIG. 8 depicts a portable positive air filtration device 300 according to another example embodiment of the present invention. In example embodiments, the device 300 is generally similar to the device 10 as described above, for example, comprising a respiratory component 312 and a harness component 350. In example embodiments, the respiratory component 312 and the harness component 350 are preferably integrally formed and integrated together for example such that the housing 320 defines a band-like unitary component (e.g., defining a ring-like profile). In example embodiments, a user U can pass their head through an opening of the housing 320 until the respiratory component 312 is engaged with the face to cover the nose and mouth thereof and with the harness component 350 placed against the head/neck area of the user U to securely position the respiratory component 312 around the mouth and nose of the user U. In example embodiments, the housing 320 defines a conduit 321 therein and which provides for fluid communication between the rear-positioned filter assembly 370 and the respiratory component 312. Thus, according to example embodiments, air is filtered by passing through the filter assembly 370, and is drawn through the conduits 321 to the respiratory component 312 for inhalation by the user U.

According to example embodiments, one or more fans 390 can be provided so as to provide a positive air flow of filtered air to the respiratory component 312. In one example embodiment, at least one fan 390 is provided within the respiratory component 312, for example, to be generally positioned near the respiratory component 312. According to another example embodiment, one or more fans 390 can be provided at other desirable locations as desired. In one example embodiment, one or more fans 390 can be positioned generally near the filter assembly 370, or for example, can be positioned within at least a portion of the conduits 321.

In example embodiments, one or more compartments 336 can be provided for housing one or more components such as a battery supply 340, an operation switch 404, a circuit board 406, a Bluetooth component, and or other hardware and/or electrical components as desired. In example embodiments, one or more sensors 420 can be provided within at least a portion of the housing 320. According to one example embodiment, the sensors 420 are photoplethysmography (PPG) sensors. In other example embodiments, the sensors 420 can comprise various other sensors, components, hardware or other componentry as desired.

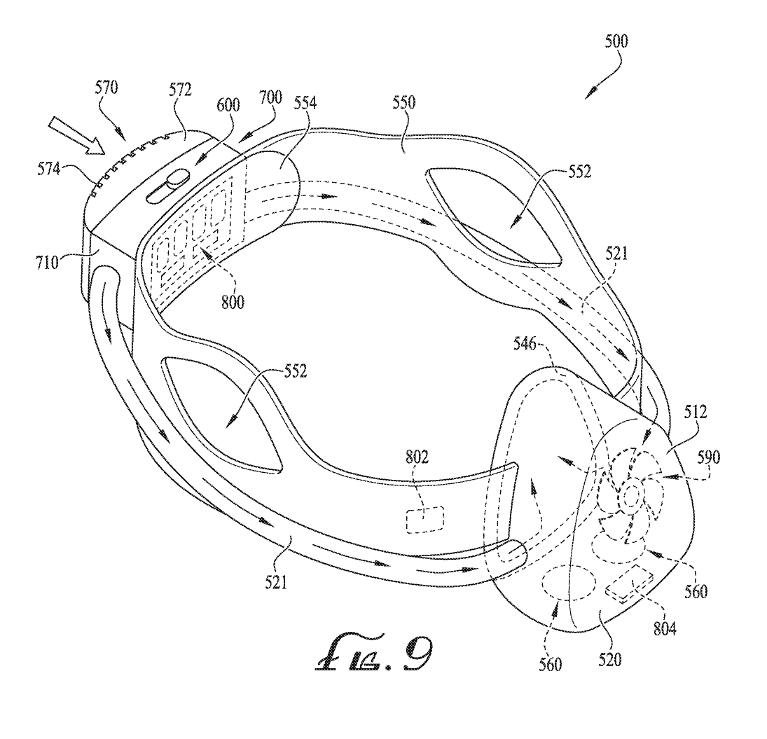

FIG. 9 shows a portable positive air filtration device 500 according to another example embodiment of the present invention. In example embodiments, the device 500 is generally similar to the device 300 as described above, for example, comprising a respiratory component 512 and a harness component 550. In example embodiments, the respiratory component 512 and the harness component 550 are integrally formed and integrated together for example such that the housing 520 defines a band-like unitary component (e.g., defining a ring-like profile) for fitting against the mouth and nose of the user U and extending around the face thereof to the rear portion of the head and neck. In example embodiments, one or more openings 552 and be formed therein. According to one example embodiment, the openings 552 can be configured so as to receive at least a portion of the ears of the user U, for example, to provide for a more comfortable and secure strapping harness so as to maintain sealed engagement of the respiratory component 512 with the face of the user U. According to example embodiments, a rear portion 554 of the harness 550 is securely repositionable to at least a portion of the harness so as to provide adjustability to the size thereof, and thus, accommodate fitting with users of all sizes. Similarly to the device 300, the filter assembly 570, operation switch 600 and housing or compartment 700 are positioned on an external portion of the harness component 550 at a side that is generally opposite the respiratory component 512.

In example embodiments, at least one conduit, hose or other tube is connected between the filter assembly 570 and the respiratory component 512 so as to allow for the air that is filtered by the filter assembly 570 to move there along and to the respiratory component 512 for inhalation by the user U. According to example embodiments, one or more fans 590 can be incorporated with the respiratory component 512, conduit 521, harness component 550, filter assembly 570 and/or compartment 770 as desired. According to the depicted embodiment, at least one fan 590 is provided within the respiratory component 512 and in fluid communication with the one or more conduits 521. In example embodiments, rotation of the fan 590 preferably causes air to be drawn from the conduits 521 and within the respiratory component 512 for inhalation by the user U. Preferably, the one or more fans 590 are capable of providing filtered air to the user U at a positive pressure. In other example embodiments, one or more fans 590 can be provided within at least a portion of the compartment 700 and/or within at least a portion of the conduits 521. In one example embodiment, one or more exhalation valves 560 can be provided so as to allow completely exhausting the exhalation of CO₂ from the respiratory component 512.

In example embodiments, the compartment 700 can be provided for housing one or more components 800, for example, such as a battery supply, an operation switch, a circuit board, a Bluetooth component, memory, sensors, controls, connectors, outlets, or for example any other components, hardware, software, etc. so as to support the entirety of the electronic control system, and for example, provide for connecting and communicating with an electronic device D (as described above) and or other hardware and/or electrical/electronic components as desired. In example embodiments, one or more sensors 802 are provided within at least a portion of the harness component 550 (e.g., PPG and/or EKG sensors according to example embodiments), and for example, one or more additional components can be housed within the respiratory component 512 or housing 520 thereof. For example according to one example embodiment, the housing 520 comprises a battery supply 804 for powering the electronic control system so as to power the fan 590 and other components 800, and for example, to provide power thereto for connecting and communicating with at least one electronic device D and/or one or more devices 500. According to some example embodiments, a battery supply is provided in the compartment 700 and the battery supply 804 is housed or contained within the housing 520.

According to some example embodiments, a charging port is provided on at least a portion of the compartment 700 such that a charging cable can connect thereto for charging the one or more of the battery supplies. According to additional example embodiments, for example, as described above, a separate or external power supply can be connected to the electronic control system and removably mounted to at least a portion of the device 500. Optionally, a charging dock can be provided such that at least a portion of the device 500 comprises a receiver for receiving a charging pin of the dock. According to some example embodiments, the electronic control system is configured such that the battery supply can be recharged by induction charging. According to some example embodiments, an induction charging dock can be provided for placement atop a table or other surface, and for example, at least a portion of the device 500 can be placed thereon such that the battery supply begins to recharge. In another example embodiment, a wall-mounted charging component can be provided such that hanging or connecting at least a portion of the device 500 with the wall-mounted component, the device 500 would remain engaged therewith (e.g., generally stored or organized on the wall) while also being electrically connected (hard wire or wireless induction) therewith such that the battery supply is recharged.

According to example embodiments, one or more of the devices 10, 300, 500 of the present invention can be provided for use with wellness patients, for example, patients that may have some kind of condition such that monitoring their respiratory rates could be advantageous for diagnosing and monitoring and tracking one or more desirable characteristics of the user's respiratory condition as they are breathing (e.g., inhaling and exhaling) while the respiratory component 512 is sealed around the nostril openings and mouth of the user. According to some example embodiments, the wellness patients are suffering from a respiratory condition or other condition such that their doctor prescribed a device 10, 300, 500 for diagnosing some of the particular concerns of the user's U condition in addition to monitoring the progress thereof. According to other example embodiments of the present invention, one or more of the devices 10, 300, 500 can be worn and used by athletes or other training platforms or programs so as to collect and process data obtained therefrom and generally saved on the server S.

According to example embodiments, one or more algorithms, scripts and/or programs of the software or application of the electronic device D (or database/server S, or other device PC) can assist with the development of a respiratory algorithm for the user U, for example, that is based on a plurality of calculations and/or data points associated with the obtained data from the electronic control system of the devices 10, 300, 500. According to one example embodiment, the respiratory algorithm can include data such as the measured heart rate, CO₂ and oxygen levels, temperatures (e.g., external, internal and/or near any electrical components/battery), pressures, direct measurement of ventilation parameters such as pulse oximetry including respiration rate and respiration volume, a quantifiable estimation of the air-flow in and out of the lungs of the user U, and other directly or indirectly obtainable or measureable data as will be described below.

According to one example embodiment, the software of the electronic device D (or other devices PC) can comprise one or more programs, scripts, algorithms or other code so as to provide the respiration performance of the user U that is wearing the device 10, 300, 500. For example, according to example embodiments, the respiration performance is generally based upon one or more parameters including the directly-measured amount of air-flow in and out of the lungs of the user U, pulse oximetry (e.g., PPG sensor), measured heart rate, CO₂ and oxygen levels, temperatures, body temperature, and/or other measurements and/or characteristics of the user U, the device 10, 300, 500, the surrounding environment, air quality, type of activity being performed during use of the device 10, 300, 500 (steps, workout, run, etc.), and/or other desired data. Thus, according to some example embodiments, the devices 10, 300, 500 as described herein preferably provide for direct ventilation measurement of respiration performance.

According to example embodiments, the actual energy consumption of the user U wearing and using the devices 10, 300, 500 of the present invention is obtainable. For example, according to example embodiments, actual energy consumption is monitored by tracking the consumption of oxygen through ventilation monitoring. For example, by use of the PPG sensor(s) and/or one or more other direct ventilation measurements (e.g., directly measured amount of air flow in and out of the lungs of the user U) can be provided so as to measure directly (and process/calculate) the amount of energy (e.g., kcal units) the user U is consuming during a given activity. According to one example embodiment, the respiration volume can be calculated by measuring the speed and electrical current of the one or more fans, providing a flow meter so as to directly obtain the exhalation of air from the user, or for example, a sensor so as to monitor the movement and time they are open during exhalation. Optionally, other components, sensors, measurements, calculations, scripts, algorithms, or other methods can be provided so as to directly measure and obtain an accurate respiration volume.

According to some example embodiments, by direct measurement of respiratory parameters, tracking and processing these parameters can determine if a wearer has gone beyond the point of exhaustion and is endangering their health. Accordingly, according to example embodiments, the device 10, 300, 500 (or electronic device D) connected therewith can alert the user U of their present respiratory parameters and recommend taking a break or pausing from the activity until the respiratory parameters fall back within normal healthy conditions. According to example embodiments, an alert is emitted from the electronic device D, or for example, an alert can be emitted from the device 10, 300, 500. According to some example embodiments, an alert from each of the electronic device D and device 10, 300, 500 can be provided. According to other example embodiments of the present invention, the software of the electronic device D can be configured so as to provide reassurance to the user U and the measured respiratory parameters. For example, according to some example embodiments, reassurance can be provided to a user U that physically show no signs of fatigue and that any weariness is psychological rather than physical. Accordingly, according to some example embodiments, one or more alerts or other notifications can be provided so as to encourage the user U to raise their level of activity, or for example, to reassure them that they are not at risk of endangerment and should not pause from a given activity based on the present respiratory parameters.

According to one example embodiment, the software of the electronic device D (or other devices PC) can comprise one or more programs, scripts, algorithms or other code so as to monitor other components and/or characteristics of the device 10, 300, 500, the working environment, the user U or other measureable data as desired. According to one example embodiment, the level of filter blockage can be determined by the speed and electrical current of the one or more fans. In example embodiments, the fan(s) are controlled to run at a fixed speed, which is measured directly from a built-in tachometer thereof, and thus, measuring the electrical current of the fan will indicate how much resistance the fan is encountering. In example embodiments, the filter porosity is the only factor affecting resistance to the fan, and therefore, if fan resistance increases then this is a direct measure of reduced filter porosity or increased filter blockage. According to example embodiments, the application of the electronic device D comprises a menu and selection of filters, for example, so that the user can assign a filter to the device 10, 300, 500, thereby allowing for accurate measurement and reporting regarding the present level of filter blockage. In other example embodiments, the user can input one or more materials, gasses, etc. which they may be exposed to while using the device 10, for example, so that the device D can recommend a particular filter based upon the selected conditions and potential exposure.

According to another example embodiment, the electronic control system of the device 10, 300, 500 can send alerts to the electronic device to indicate if a filter is becoming blocked more quickly than expected, and for example, to make recommendations about filter pore size. According to example embodiments, the electronic device D can provide users with automated feedback about the particulates in the atmosphere that they are breathing, or for example, that they are not breathing and being filtered out by the filter. According to example embodiments, information pertaining to the atmospheric conditions is determined via a third-party input to the software of the application of the electronic device D, and thus, via the electronic device's D location (via GPS of the device D), the application can be provided with a third-party provider's current atmospheric conditions of the GPS's location. Optionally, one or more measuring devices and/or other hardware/software can be incorporated within at least a portion of the device 10, 300, 500 so as to measure one or more qualities or characteristics of the atmospheric conditions of the location of the device.

According to example embodiments, providing the user U with the current atmospheric conditions of the current location can be valuable and desirable for sufferers of respiratory conditions. In their case it may not be possible to permit larger particulates through a larger pore size filter grade; however it may be necessary to either warn such users U to avoid outdoor conditions temporarily until the pollution is reduced or to advise such users to switch to a larger surface-area filter. Preferably, the electronic control system provides automatic feedback about the effectiveness and selection of the filter. According to another example embodiment, the device offers audible alerting means that irrespective of the software application of the electronic device D, the device 10, 300, 500 can offer customizable stand-alone alerts to the wearer or user.

In other example embodiments, respiratory performance can be measured during the training and/or practicing of athletes. In example embodiments, the athlete's respiratory performance is collected and processed. Preferably, monitoring an athlete's respiratory performance can provide beneficial data and feedback concerning the health and potential of an athlete. In other example embodiments, a plurality of athletes can be monitored together in real time.

While the invention has been described with reference to example embodiments, it will be understood by those skilled in the art that a variety of modifications, additions and deletions are within the scope of the invention, as defined by the following claims.

What is claimed is:

1. A portable positive air filtration device comprising:
a respiration component for covering the mouth and nostril of a user and a harness component for attachment to the respiration component so as to maintain sealed engagement of the respiration component around the mouth and nostril,
wherein the respiration component comprises at least one fan or air-moving component, an air filter assembly, an electronics control module electrically connected with the at least one fan, a battery supply for powering the at least one fan and electronics control module such that filtered air can be provided to the mouth and nostril of the user at a positive pressure.

2. The portable positive air filtration device of claim 1, further comprising a seal or interface provided on at least a portion of the respiratory component for providing a seal against the user's face when the respiration component is placed thereagainst and surrounding the mouth and nostril thereof.

3. The portable positive air filtration device of claim 1, further comprising one or more one-way valves so as to permit exhausting carbon dioxide from the respiratory component.

4. The portable positive air filtration device of claim 1, further comprising an operation switch for turning on and off the electronics control module and at least one fan.

5. The portable positive air filtration device of claim 1, further comprising an electronic device for connecting and communicating with the electronics control module, wherein data of one or more components of the electronics control module or other measurements or characteristics thereof can be sent to the electronic device so as to be collected and further processed.

6. The portable positive air filtration device of claim 5, further comprising sending the data to a database or server over a network.

7. The portable positive air filtration device of claim 5, wherein the electronics control module and the electronic device communicate by Bluetooth connection.

8. The portable positive air filtration device of claim 5, wherein one or more alerts or notifications can be displayed or emitted from the electronic device and/or the electronics control module, the alerts or notifications based on one or more measurements or data that has been determined to be outside of the normal operating conditions.

9. The portable positive air filtration device of claim 1, wherein one or more ventilation parameters can be measured and processed so as to allow for the development of a respiratory algorithm in determining the respiratory performance of a user wearing and operating the portable positive air filtration device.

10. The portable positive air filtration device of claim 9, further comprising a photoplethysmography sensor so as to utilize pulse oximetry for determining the user's present respiratory status and condition.

11. The portable positive air filtration device of claim 9, further comprising one or more components, hardware or software so as to directly measure the volume of air inhaled and exhaled from the lungs of the user.

12. The portable positive air filtration device of claim 11, wherein the measured volume of air inhaled and exhaled from the lungs of the user can be processed so as to output the actual energy consumption by a user during a given activity.

13. A portable positive air filtration system comprising:
one or more portable powered respiratory devices, the respiratory devices comprising a mask portion for covering the mouth and nostril of a user, a filter, a battery supply, and one or more fans for providing filtered oxygen to the mouth and nostril of the user at a positive pressure;
an electronics control module integrated in each of the one or more powered respiratory devices, the electronics control module comprising one or more electrical components, hardware, software, sensors, a processor and/or memory;
at least one electronic device for connecting and communicating with the electronics control module of the one or more powered respiratory devices.

14. The portable positive air filtration system of claim 13, wherein the respiratory device is portable and wearable by the user without being cumbersome and obtrusive, thereby permitting the user to wear and utilize the device in any environment and/or while performing any activity, sport or other body movements.

15. The portable positive air filtration system of claim 13, further comprising a server that is connectable with the electronic device by a network, wherein any and all data obtainable by the electronics control module can be sent to the electronic device for collection and processing, and where the data can further be sent to the server for collection and processing, wherein the server is accessible by one or more electronic devices.

16. The portable positive air filtration system of claim 13, wherein the electronics control module and/or electronic device can output a direct ventilation measurement of respiration performance by measuring a respiration rate and a respiration volume in real time.

17. The portable positive air filtration system of claim 16, wherein the respiration rate can be measured by use of a photoplethysmography sensor that is integrated in the respiratory device, and wherein the respiration volume can be obtained by directly measuring the volume of air that is inhaled and exhaled by the user wearing and operating the respiratory device.

18. The portable positive air filtration system of claim 17, wherein data associated with the respiration performance including the respiration rate and respiration volume can be accessed on the electronic device, server or one or more other electronic devices.

19. The portable positive air filtration system of claim 18, wherein one or more alerts or notifications can be emitted from the electronic device, the one or more respiratory devices, or one or more other electronic devices when a calculated value of the respiration performance falls below or exceeds values within normal operating conditions.

20. A positive air filtration device comprising a respiratory component and a harness component connected with the respiratory component, wherein at least one of the components comprises one or more fans, one or more filters, one or more battery supply components, and an electronic control module comprising a plurality of electronic components including a processor and memory, a circuit board, and a connection component for connecting and communicating with one or more electronic devices comprising a processor, memory, software and an application for displaying, processing and/or calculating data that is provided from the electronic control module of the positive air filtration device, the respiratory component comprising an interface portion for covering the mouth and nostril of a user and wearer of the device such that filtered oxygen can be provided to the mount and nostril of a user at a positive pressure.

21. The positive air filtration device of claim 20, wherein the respiratory component and the harness component are integrally formed together.

22. The positive air filtration device of claim 20, wherein the respiratory component and the harness component are separate pieces and removably or permanently attached to the respiratory component.

23. The positive air filtration device of claim 20, wherein the harness component is adjustable

24. The positive air filtration device of claim 20, wherein the connection component is a Bluetooth component.
